(19) United States
(12) Patent Application Publication
(10) Pub. No.: US 2023/0027482 A1
(43) Pub. Date: Jan. 26, 2023

(54) METHOD FOR PRODUCING DI- AND POLYISOCYANATES OF THE DIPHENYLMETHANE SERIES

(71) Applicants: Covestro Deutschland AG, Leverkusen (DE); Covestro Intellectual Property GmbH & Co. KG, Leverkusen (DE)

(72) Inventors: Stefan Wershofen, Mönchengladbach (DE); Enke Liu, Leverkusen (DE); Anke Hielscher, Köln (DE); Richard Adamson, Leichlingen (DE); Ricardo Serra, Erkrath (DE); Volker Michele, Köln (DE); Juergen Spriewald, Köln (DE)

(21) Appl. No.: 17/783,367

(22) PCT Filed: Dec. 15, 2020
(86) PCT No.: PCT/EP2020/086261

§ 371 Date: Jun. 8, 2022

(30) Foreign Application Priority Data
Dec. 18, 2019 (EP) 19217601.4
Oct. 29, 2020 (EP) 20204702.3

(51) Int. Cl. C07C 263/10

(52) U.S. Cl. CPC C07C 263/10

(57) ABSTRACT

The invention relates to a method for producing di- and polyisocyanates of the diphenylmethane series, in which (i) the liquid product flow created in the phosgenation, (ii) the reaction mixture present in an optionally provided reactor for carbamic acid chloride cleaving, (ii) the liquid product flow leaving such an optionally provided reactor for carbamic acid chloride cleaving, (iv) the reaction mixture present in the dephosgenation, or (v) the liquid product flow created in the dephosgenation, is treated with a gaseous hydrogen chloride flow in one stage in a bubble column or in a plate column within a contact time of 1 min to less than 30 min. The product flow treated din this way or in the reaction mixture treated in this way is fed directly (i.e. in particular without further treatment with an inert gas) to the next step of the reaction or workup.

The invention relates to a process for preparing di- and polyisocyanates of the diphenylmethane series, wherein
(i) the liquid product stream obtained in the phosgenation, (ii) the reaction mixture present in an optionally present reactor for carbamoyl chloride cleavage, (iii) the liquid product stream exiting such an optionally present reactor for carbamoyl chloride cleavage, (iv) the reaction mixture present in the dephosgenation or (v) the liquid product stream obtained in the dephosgenation,
   is treated with a gaseous hydrogen chloride stream over a contact time of 1 min to less than 30 min in a single stage in a bubble column or in a tray column. The product stream treated in this way or the reaction mixture treated in this way is supplied directly (i.e. in particular without further treatment with an inert gas) to the next step of the reaction or workup.

Aromatic di- and polyisocyanates are important and versatile raw materials for polyurethane chemistry. In addition to tolylene diisocyanate, the di- and polyisocyanates of the diphenylmethane series (henceforth referred to collectively as MDI) are of particular industrial interest. In all production processes relevant on a large industrial scale, MDI is obtained by phosgenation of the corresponding di- and polyamines of the diphenylmethane series (henceforth referred to collectively as MDA). MDA in turn is prepared by acid-catalyzed reaction of aniline and formaldehyde.

A recurring problem encountered in connection with the preparation or storage of MDI is the dark coloring thereof. There has been no lack of attempts to prevent this and to provide an MDI that is as “light-colored” as possible. For instance, Japanese patent application JP 2010/120870 A describes a process for preparing MDI by phosgenation of MDA in an organic solvent, wherein, after complete phosgenation and separation of a substantial portion of the solvent employed (down to a residual concentration in the range from 5% to 35% by mass) and thus also of the phosgene, hydrogen chloride gas is added to the reaction mixture, wherein the temperature during the phosgenation, the solvent separation and the hydrogen chloride treatment is maintained in a range from 60° C. to 130° C. However, treatment with hydrogen chloride gas after separation of a substantial portion of the solvent has the disadvantage that the mixture to be treated is more difficult to handle due to a relatively high viscosity. In addition, a reduction in the efficiency of the treatment and increased formation of deposits would also be anticipated.

Japanese patent application JP2010018534 (A) describes a process in which an aromatic polyamine is first obtained by acid-catalyzed reaction of aniline and formaldehyde or paraformaldehyde in an ionic liquid followed by extraction with a hydrophobic organic solvent. Phosgenation of the aromatic polyamine prepared in this way and separation of solvent and/or excess phosgene affords a first isocyanate. This first isocyanate is then mixed at 60° C. to 160° C. with a hydrogen chloride-containing second isocyanate obtained by incorporation of hydrogen chloride into an isocyanate end product prepared separately (especially commercially available MDI). The mixture obtained is then heated to temperatures in the range from 180° C. to 230° C. Japanese patent JP 5158199 B2 relates to a similar process. These procedures deliberately avoid entry of hydrogen chloride gas into the actual MDI production process. They suffer from considerable disadvantages such as, for instance, the use of ionic liquids, which is costly and complex.

Japanese patent application JP H07233136 A is concerned with improving the color of MDI and also, in particular, with reducing the content of so-called hydrolyzable chlorine in MDI. To this end, a plurality of apparatuses for treating the crude MDI with hydrogen chloride at elevated temperature are connected between the dephosgenation column (for removing excess phosgene) and the solvent separation column (for removing the solvent employed). In this multi-stage hydrogen chloride treatment performed after the dephosgenation the temperature increases from stage to stage, in particular from 60° C. to 140° C. in a first stage (for example 30 min at 115° C.) to 150° C. to 170° C. in a second stage (for example 3 min at 160 ° C.). The multi-stage hydrogen chloride treatment described is complex and requires capital expenditure on several additional apparatuses. A single-stage hydrogen chloride treatment after the dephosgenation is portrayed as disadvantageous (see comparative example 1). A single-stage hydrogen chloride treatment without a separate dephosgenation (i.e. this hydrogen chloride treatment is to effect not only lightening but also stripping of phosgene) is likewise portrayed as disadvantageous (see comparative example 2).

Japanese patent application JP 2004/027160 A describes a process for preparing MDI which comprises passing initially hydrogen chloride gas and then an inert gas such as nitrogen through a crude MDI solution. In all specifically disclosed embodiments, in particular also in all examples (and in all comparative examples), the crude MDI solution treated with hydrogen chloride gas is a solution obtained by dephosgenation of the reaction product of the phosgenation of MDA with phosgene in the presence of a solvent. The inert gas treatment may be carried out either immediately after the treatment with hydrogen chloride gas or after an intermediate solvent separation. This reduces the content of hydrolyzable chlorine in the MDI. The document explicitly concludes that a hydrogen chloride treatment on its own, without subsequent inert gas treatment, is disadvantageous. It must therefore be assumed that process parameters of the hydrogen chloride treatment disclosed in the document relate to the claimed combined process comprising a treatment with hydrogen chloride and inert gas. However, the additional treatment with an inert gas is complex, generates costs and results in an elevated offgas load.

Japanese patent application JP 2006/104166 A describes a method for reducing the content of hydrolyzable chlorine, wherein crude MDI (which has only been subjected to a separation of solvent and excess phosgene) has a gas passed through it at a temperature in a range from 150° C. to 250° C. at a pressure of 5 to 25 kPa in the presence of a metal oxide. Nitrogen, hydrogen chloride, hydrogen bromide, carbon monoxide and carbon dioxide are described as suitable gases. The use of metal oxides is complex and generates costs. The metal oxides must also be separated off again.

German patent application DE 28 47 243 A1 describes a process for preparing an organic isocyanate, wherein an organic amine is reacted with phosgene to provide a reaction product which contains the corresponding organic isocyanate, wherein hydrogen chloride gas is passed through this reaction product in the presence of an inert organic solvent, wherein the gas present in this reaction product is removed and the acidic and hydrolyzable chlorine-containing substances present in the reaction product are minimized The reaction product is the crude liquid process product of the reaction of the amine with phosgene (which is carried out in the presence of a solvent). The gas phase likewise formed during the reaction, which consists essentially of hydrogen chloride gas and unconverted phosgene, may, once excess phosgene has been largely absorbed in a solvent, be used directly for the purpose of passing hydrogen chloride gas through the reaction product. This strips phosgene still dissolved in the reaction product. A separate step of dephosgenation distinct from the hydrogen chloride treatment is not described. (Only the prior-art stripping of dissolved phosgene by passing through nitrogen, from which the process described in DE 28 47 243 A1 seeks to distinguish itself, is described.) The hydrogen chloride gas treatment is preferably carried out in a random-packed column at a temperature of 140° C. to 230° C. over a duration of 0.5 to 5 hours. Other reaction apparatuses or shorter contact times are not disclosed in the document.

As described, the processes of the prior art, which aim to improve the color (and/or reduce the content of hydrolyzable chlorine) of the MDI, have various disadvantages. There was therefore still a need for improvements in this field. It would in particular be desirable to have available an efficient method for lightening the color of MDI which may be integrated into existing MDI production plants with as little apparatus complexity as possible, which causes as little additional offgas load as possible and which can dispense with the use of catalysts foreign to the system (such as metal oxides). It would moreover be particularly desirable to provide an efficient method for lightening the color and/or reducing the chlorine content (expressed as total chlorine content and/or as hydrolyzable chlorine) of MDI which requires as few additional reagents as possible.

Taking this requirement into account the present invention provides a process for preparing di- and polyisocyanates of the diphenylmethane series, comprising the steps of:

—Reaction—
(I) reacting di- and polyamines of the diphenylmethane series with phosgene in the presence of a solvent in a phosgenation reactor to obtain (a) a first gaseous product stream containing hydrogen chloride and phosgene (and optionally evaporated solvent) and (b) a first liquid product stream containing di- and polyisocyanates of the diphenylmethane series, phosgene and solvent (and optionally also carbamoyl chlorides); and
(II) optionally reacting the first liquid product stream in a reactor for carbamoyl chloride cleavage to form in the reactor for carbamoyl chloride cleavage a first reaction mixture which is separated into (a) a second gaseous product stream containing hydrogen chloride and phosgene (and optionally evaporated solvent) and (b) a second liquid product stream containing isocyanate, phosgene and solvent; and

—Workup—
(III) separating phosgene from the first liquid product stream or, if step (II) is performed, from the second liquid product stream in a dephosgenation apparatus to form in the dephosgenation apparatus a second reaction mixture which is separated into (a) a third gaseous product stream containing hydrogen chloride and phosgene (and optionally evaporated solvent) and (b) a third liquid product stream containing isocyanate and solvent (which is depleted in phosgene relative to the first or second liquid product stream);
(IV) separating solvent from the third liquid product stream to obtain (a) a fourth gaseous product stream containing solvent and (b) a fourth liquid product stream containing isocyanate, wherein the separation of the solvent is not followed by an inert gas treatment of the fourth liquid product stream obtained;
(V) optionally separating a portion of the diisocyanates of the diphenylmethane series from the fourth liquid product stream to obtain (a) a fifth gaseous product stream containing diisocyanates of the diphenylmethane series, which is condensed, and (b) a fifth liquid product stream enriched in polyisocyanates of the diphenylmethane series relative to the fourth liquid product stream;
(VI) optionally separating phosgene from the first and third gaseous product stream, if performing step (II) from the first, second and third gaseous product stream, to obtain (a) a sixth gaseous product stream containing hydrogen chloride and (b) a sixth liquid product stream containing phosgene (and solvent if such solvent was present in the gaseous product streams);
   wherein
(VII) (a) the first liquid product stream obtained in step (I) is treated with a gaseous hydrogen chloride stream and then supplied directly to step (II) or step (III) or
   (b) the first reaction mixture formed in the reactor for carbamoyl chloride cleavage from step (II) is treated with a gaseous hydrogen chloride stream or
   (c) the second liquid product stream obtained in step (II) is treated with a gaseous hydrogen chloride stream and then supplied directly to step (III) or
   (d) the second reaction mixture formed in the dephosgenation apparatus from step (III) is treated with a gaseous hydrogen chloride stream or
   (e) the third liquid product stream obtained in step (III) is treated with a gaseous hydrogen chloride stream and then supplied directly to step (IV),
      wherein the treatment with the gaseous hydrogen chloride stream is performed in a single stage in a bubble column or in a tray column, wherein a contact time of the gaseous hydrogen chloride stream with the (first, second or third) liquid product stream to be treated or the (first or second) reaction mixture to be treated in the range from 1 min to less than 30 min is established.

According to the invention a gaseous hydrogen chloride stream is to be understood as meaning a gas stream which comprises hydrogen chloride in a proportion based on its total mass of at least 90% by mass, preferably at least 95% by mass, particularly preferably at least 98% by mass and very particularly preferably at least 99% by mass. It may be a pure gaseous stream of hydrogen chloride, i.e. a stream which comprises only hydrogen chloride gas, optionally apart from insignificant trace amounts of impurities resulting from production, which, however, do not regularly exceed 2 000 ppm and are therefore negligible. However it may also be a gaseous hydrogen chloride stream which comprises foreign substances, in particular phosgene, gases solvents and/or inert gases, in significant proportions, for example a hydrogen chloride stream obtained in the process according to the invention itself. However, such a proportion of foreign substances accounts for not more than 10% by mass, preferably not more than 5% by mass, particularly preferably not more than 2% by mass and very particularly preferably not more than 1% by mass, based on the total mass of the gaseous hydrogen chloride stream.

The treatment “with a gaseous hydrogen chloride stream” (henceforth also referred to as hydrogen chloride treatment according to the invention for short) to be undertaken in step (VII) according to the invention may in principle be performed with any product stream or reaction mixture mentioned under (VII) (a) to (VII) (e). In the context of the present invention the expression “is treated with a gaseous stream of hydrogen chloride” describes passing hydrogen chloride gas through the product stream to be treated or the reaction mixture to be treated.

The hydrogen chloride treatment according to the invention is carried out “in a single stage”, i.e. the product stream to be treated or the reaction mixture to be treated having a particular temperature has hydrogen chloride gas of a particular temperature passed through it continuously in one step (and not as in some processes of the prior art in a plurality of steps in serially arranged apparatuses with successively increasing temperature).

In contrast to some prior art processes, the hydrogen chloride treatment according to the invention does without the addition of catalysts (such as metal oxides); step (VII) is therefore performed especially without addition of a catalyst.

After performance of the hydrogen chloride treatment according to the invention the product stream treated in this way or the reaction mixture treated in this way is supplied directly to the next step of the reaction or workup, wherein “supplied directly” is to be understood as meaning that no further intermediate process steps are performed. In the case of the hydrogen chloride treatment of the first, second or third liquid product stream, the next step is step (II), (III) or (IV) respectively. In the case of the hydrogen chloride treatment of the first or second reaction mixture, the next step is a substep of step (II) or (III), namely the separation of the first or second reaction mixture into a gaseous phase and a liquid phase. Accordingly, if the hydrogen chloride treatment according to the invention is carried out on the first liquid product stream according to variant (VII)(a) for example, this stream is supplied directly (or immediately; i.e. in particular without further treatment with an inert gas as described in some processes of the prior art) to step (II) or—if this optional step is omitted—to step (III) after the hydrogen chloride treatment according to the invention. In this context the terminology used in the present invention does not particularly distinguish between a product stream (or reaction mixture) which is untreated (without or before hydrogen chloride treatment according to the invention) and one which is treated (after hydrogen chloride treatment according to the invention); both may be referred to as “first liquid product stream” for example.

It goes without saying that variants (VII)(b) and (VII)(c) are applicable only when step (II) is performed. However, performance of step (II) does not necessarily imply the use of one of these variants. It is quite possible to perform step (II) and perform the hydrogen chloride treatment according to (VII)(a) for example.

A “bubble column”(also known as a bubble column reactor) is in the context of the present invention in line with common usage in the art to be understood as meaning a process engineering apparatus for gas/liquid processes, wherein in the bubble column a gas (here: the gaseous hydrogen chloride stream) is introduced into a liquid (here: the product stream to be treated or the reaction mixture to be treated) and passes through the liquid in the form of bubbles to generate an interface between the gas and the liquid. The gas is preferably supplied through a perforated base plate through which the gas flows. The liquid can be run in cocurrent or in countercurrent.

A “tray column” is in the context of the present invention in line with common usage in the art to be understood as meaning a distillation column comprising horizontal internals, so-called trays. The trays are plates having openings. Suitable trays of this kind are so-called sieve trays, bubble-cap trays or valve trays on which liquid (here: the product stream to be treated or the reaction mixture to be treated) collects. A gas phase (here: the gaseous hydrogen chloride stream) is passed into the liquid through special slots or holes, thus forming a bubble layer. A new temperature-dependent equilibrium between the liquid phase and the gas phase is established on each of these trays. One tray ideally corresponds to one theoretical separation plate.

In the appended drawings:

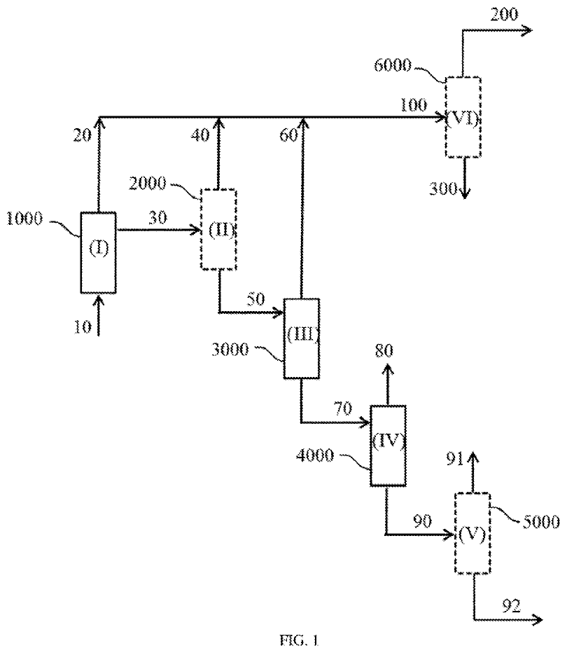

FIG. 1 shows a representation of steps (I) to (VI) of the process according to the invention;

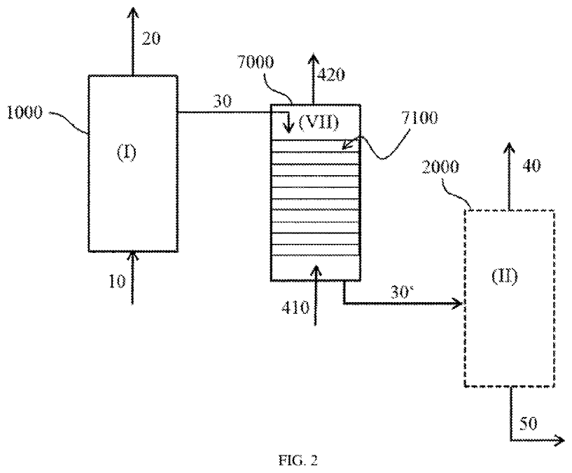

FIG. 2 shows a representation of a preferred implementation of step (VII) of the process according to the invention.

There follows firstly a brief summary of various possible embodiments.

In a first embodiment of the invention, step (VII) is carried out according to variant (VII)(a), (VII)(c) or (VII)(e).

In a second embodiment of the invention, step (VII) is carried out according to variant (VII)(a) or (VII)(c).

In a third embodiment of the invention which may be combined with all other embodiments, the third liquid product stream contains phosgene in a mass fraction based on its total mass in the range from 0.001 ppm to 1000 ppm, preferably in the range from 0.01 ppm to 100 ppm.

In a fourth embodiment of the invention which may be combined with all other embodiments, the gaseous hydrogen chloride stream employed in step (VII) contains phosgene in a mass fraction based on its total mass in the range from 1 ppm to 10 000 ppm, preferably 10 ppm to 10 000 ppm, particularly preferably in the range from 10 ppm to 1000 ppm.

In a fifth embodiment of the invention which may be combined with all other embodiments, in particular with the fourth embodiment, the process comprises step (VI), wherein the sixth gaseous product stream is partially or completely used as the gaseous hydrogen chloride stream in step (VII) (to which further hydrogen chloride may be but need not be added).

In a sixth embodiment of the invention which may be combined with all other embodiments, the molar ratio of hydrogen chloride present in the gaseous hydrogen chloride stream employed in step (VII) to isocyanate groups present in the reaction mixture or product stream treated in step (VII), n(HCl)/n(NCO), is set to a value in the range from 0.1 to 2.0, preferably 0.1 to 1.0, particularly preferably 0.1 to 0.5.

In a seventh embodiment of the invention which may be combined with all other embodiments provided these are not limited to a cocurrent mode of operation in step (VII) (i.e. with the exception of the eighth embodiment described below),
the gaseous hydrogen chloride stream and
the product stream to be treated or the reaction mixture to be treated
   are run in countercurrent in step (VII).

In an eighth embodiment of the invention which may be combined with all other embodiments provided these are not limited to a countercurrent mode of operation in step (VII) (i.e. with the exception of the seventh embodiment),
the gaseous hydrogen chloride stream and
the product stream to be treated or the reaction mixture to be treated are run in cocurrent in step (VII).

In a ninth embodiment of the invention which may be combined with all other embodiments, the treatment with the gaseous hydrogen chloride stream in step (VII) is performed at a temperature of the product stream to be treated or of the product mixture to be treated in the range from 70° C. to 135° C., preferably 80° C. to 135° C., particularly preferably 90° C. to 135° C., and at a temperature of the hydrogen chloride gas stream employed in the range from 20° C. to 135° C., preferably 25° C. to 135° C., particularly preferably 30° C. to 135° C.

In a tenth embodiment of the invention which may be combined with all other embodiments, in particular with the twelfth embodiment, the treatment with the gaseous hydrogen chloride stream in step (VII) is performed isothermally.

In an eleventh embodiment of the invention which may be combined with all other embodiments, a contact time of the gaseous hydrogen chloride stream with the (first, second or third) liquid product stream to be treated or the (first or second) reaction mixture to be treated in the range from 1 min to 25 min, preferably 1 min to 20 min, particularly preferably 2 min to 20 min, very particularly preferably 4 min to 20 min, is established in step (VII).

In a twelfth embodiment of the invention which may be combined with all other embodiments, the treatment with the gaseous hydrogen chloride stream in step (VII) is carried out at a pressure in the range from ambient pressure to 5.00 bar_{(abs)}, preferably at a pressure in the range from 1.50 bar_{(abs.)} to 3.50 bar_{(abs).}

The embodiments briefly outlined above and further possible configurations of the invention are elucidated more particularly hereinbelow. The specified embodiments and further possible configurations may be combined with one another as desired unless the opposite is apparent from the context.

The actual preparation of the MDI according to steps (I) to (VI) may be effected as is known per se from the prior art, provided that the hydrogen chloride treatment according to the invention according to step (VII) is ensured. FIG. 1 shows a production plant suitable therefor in a schematic and greatly simplified form. The reference symbols are defined as follows.

Apparatuses (apparatuses optional in the broadest embodiment of the invention are shown with dashed lines):
1000 phosgenation reactor;
2000 reactor for carbamoyl chloride cleavage (optional);
3000 dephosgenation apparatus;
4000 apparatus for solvent separation;
5000 apparatus for partial separation of diisocyanates of the diphenylmethane series (optional and preferred);
6000 apparatus for separation of phosgene from phosgene-containing gas streams obtained in the process (optional and preferred).

Material Streams:
10 mixture of MDA, phosgene and solvent;
20 first gaseous product stream;
30 first liquid product stream;
40 second gaseous product stream;
50 second liquid product stream;
60 third gaseous product stream;
70 third liquid product stream;
80 fourth gaseous product stream;
90 fourth liquid product stream;
91 fifth gaseous product stream;
92 fifth liquid product stream;
100 mixture of the first, second (if present) and third gas stream;
200 sixth gaseous stream;
300 sixth liquid stream.

The production of MDI may be summarized exemplarily as follows:
a) Core operation of step (I): In the phosgenation reactor (1000) MDA is reacted with phosgene in a solvent to afford MDI. To this end, solutions of MDA in the solvent and of phosgene in the solvent are preferably initially prepared and subjected to mixing in the phosgenation reactor or a mixing apparatus arranged upstream thereof. The crude process product obtained in the phosgenation reactor is separated into a liquid stream (30) comprising crude MDI and solvent (and also phosgene and hydrogen chloride) and a gaseous stream (20) comprising phosgene and hydrogen chloride (and also solvent). Various reactor types are suitable as phosgenation reactors, for example tower reactors (also known as phosgenation towers), tubular reactors or stirred tanks. It is possible and does not depart from the scope of the present invention to connect a plurality of phosgenation reactors in series and/or parallel, for example a series connection in the form of a stirred tank cascade.
b) Core operation of step (II): If required, the stream 30 obtained in the phosgenation reactor is subjected to further reaction to eliminate hydrogen chloride (discharged via stream 40) in a reactor for carbamoyl chloride cleavage (2000) (the so-called CC cleaver). This step may be performed when the stream 30 obtained in the phosgenation reactor still contains substantial proportions of carbamoyl chloride. Step (II) provides a liquid stream 50 which has been largely to completely freed of carbamoyl chloride.
c) Core operation of step (III): Separation of further hydrogen chloride formed during the reaction together with unconverted phosgene (discharged via stream 60) from stream 30 or stream 50 in the dephosgenation apparatus (3000) (the so-called dephosgenator). This affords a liquid MDI stream 70 which has been largely to completely freed of hydrogen chloride and phosgene.
d) Core operation of step (IV): Separation of solvent from the liquid stream 70 obtained in the dephosgenation apparatus (3000) in the apparatus for solvent separation (4000) (the so-called solvent column). This affords a crude MDI stream 90 largely to completely freed of solvent. The solvent separated off is obtained initially in gaseous form as stream 80.
e) Core operation of step (V): If desired, partial separation of diisocyanates of the diphenylmethane series (henceforth also referred to as MMDI) from the crude MDI stream 90 obtained in the apparatus for solvent separation (4000) in the apparatus for partial separation of diisocyanates of the diphenylmethane series (5000) (the so-called polymer separation). This affords an MMDI stream (91) initially obtained in gaseous form and a liquid MDI stream (which is depleted in MMDI relative to stream 90).
f) Core operation of step (VI): Preferably separation of phosgene from the phosgene-containing gas streams obtained in the process (in FIG. 1 the streams 20, 40 and 60) in the apparatus for separation of phosgene (6000). If the stream 80 obtained in the solvent separation still contains excessive amounts of phosgene and hydrogen chloride it is preferable, after condensation, to subject this stream 80 to a stripping and likewise supply the stripping gas stream thus obtained to the apparatus for separation of phosgene (6000). Step (VI) affords a solvent-containing phosgene stream (300) and a hydrogen chloride-containing gas stream (200). This HCl-phosgene separation may be carried out by absorption in a solvent or (after prior liquefaction) by distillation.

The continuous production of the MDI in a) is carried out in a reaction zone by a process known from the prior art. Suitable processes are described, for example, in EP 2 077 150 B1, EP 1 616 857 A1, EP 1 873 142 A1, EP 0 716 079 B1 or EP 0 314 985 B1. However, concentrations and flow rates of the reactants MDA and phosgene are preferably chosen such that a molar ratio of phosgene to primary amino groups of 1.1:1 to 30:1, particularly preferably of 1.25:1 to 3:1, is established in the mixing zone. All processes for the production of MDI afford a crude process product which decomposes/is separated into a liquid phase (30) containing not only the desired isocyanate but also dissolved hydrogen chloride, excess dissolved phosgene and solvent, and a gas phase (20) containing hydrogen chloride gas, excess gaseous phosgene and gaseous solvent. The invention also encompasses an embodiment in which the crude isocyanate stream 30 obtained is passed through an apparatus for cleavage of carbamoyl chloride in b) prior to further processing in c).

The further separation of hydrogen chloride and phosgene from the liquid crude isocyanate stream 30 or 50 in the so-called dephosgenation apparatus in c) may be carried out by any desired process known from the prior art, for example as described in EP 1 854 783 A2, WO 2004/031132 A1 or WO 2004/056756 A1. The dephosgenation is preferably carried out by distillation in a column, the so-called dephosgenation column The third gaseous product stream is obtained at the top of the dephosgenation column, while the third liquid product stream is obtained at the bottom. The dephosgenation according to step (III) is preferably configured such that the third liquid product stream exiting the dephosgenation apparatus contains phosgene in a mass fraction based on its total mass in the range from 0.001 ppm to 1000 ppm, preferably in the range from 0.01 ppm to 100 ppm. The process modes outlined above make this easily possible for those skilled in the art.

The further separation of solvent from liquid isocyanate stream 70 thus obtained in the apparatus for solvent separation in d) may be effected by any desired process known from the prior art, preferably as described in EP 1 854 783 B1.

The optional separation of diisocyanates of the diphenylmethane series from the liquid isocyanate stream 90 obtained in step (IV) in e) may be effected by any desired process known from the prior art. Suitable processes are described in EP 1 854 783 A2 and EP 1 506 957 A1 for example, or else in EP 1 475 367 A1 or WO 2012/065927 A1.

The HCl-phosgene separation in f) may be effected by any desired process known from the prior art, preferably as described in DE-A-10260084, EP 1 849 767 A1 or EP 2 093 215 A1.

The hydrogen chloride treatment according to the invention according to step (VII) comprises one of the possible variants (a) to (e). Variant (VII)(a) is preferred. Performing the hydrogen chloride treatment according to the invention at this point in the process has a number of advantages. In the case of treatment of the first liquid product stream the thermal pre-load on the material to be treated is relatively at its lowest, thus reducing the tendency for secondary reactions during the hydrogen chloride treatment which can lead to undesired (in some cases also color-imparting) byproducts. Color improvement and reduction of the chlorine content in the end product are particularly pronounced in this embodiment. Furthermore, treatment of the first liquid product stream with hydrogen chloride causes at least a portion of the phosgene dissolved in this stream to be stripped out, thus at least partially relieving subsequent process steps of the task of phosgene separation.

If, when performing step (VII) in the preferred variant (a), step (I) employs n phosgenation reactors connected in parallel, wherein n is a natural number in the range from 2 to 10, so that a plurality of liquid product streams (30-1, 30-2, . . . 30-n) of the type of the first liquid product stream (30) are obtained, these liquid product streams (30-1, 30-2, . . . 30-n) may be combined before performing step (VII). However, it is also possible to subject these product streams to step (VII) separately, especially when the individual liquid product streams (30-1, 30-2, . . . 30-n) differ with regard to their isomer and/or homolog distribution as is not unusual in day-to-day operation of an MDI production plant. It goes without saying that it is also possible to consolidate the n liquid product streams into groups which may each be treated differently. For example in the case of six phosgenation reactors connected in parallel, i.e. n=6, the liquid product streams 30-1 and 30-2 on the one hand and the liquid product streams 30-3 and 30-4 on the other hand may each be supplied to step (VII) together while the liquid product streams 30-5 and 30-6 are each supplied to step (VII) individually. The restriction to a single variant thus does not necessarily preclude provision of several apparatuses for performing step (VII) because each of the streams (30-1, 30-2, . . . 30-n) is in any case treated with hydrogen chloride in a single stage within the meaning of the invention.

If the hydrogen chloride treatment according to the invention concerns a reaction mixture present in an apparatus (variants (VII)(b) and (VII)(d)), the hydrogen chloride stream is introduced directly into this apparatus (for example into the lower portion of the dephosgenation apparatus). In this case the corresponding apparatus (reactor for carbamoyl chloride cleavage or dephosgenation apparatus) is according to the invention then to be configured as a bubble column or a tray column. Furthermore, the reaction conditions (in particular temperature and pressure) are then to be chosen such that the processes that necessarily proceed simultaneously in this embodiment (carbamoyl chloride cleavage and hydrogen chloride treatment in variant (VII)(b) and dephosgenation and hydrogen chloride treatment in variant (VII)(d)) can proceed over the contact time for the hydrogen chloride treatment that is provided for according to the invention. It is easy for those skilled in the art to find corresponding configuration criteria. If the hydrogen chloride treatment according to the invention concerns a liquid product stream exiting an apparatus (variants (VII)(a), (VII)(c) and (VII)(e)), the hydrogen chloride treatment according to the invention is carried out in an apparatus specifically provided for this purpose, which is arranged between the apparatus from which the product stream to be treated exits and the apparatus of the next step. The apparatuses of this kind for treatment (=passing through) of a liquid (here: the corresponding liquid product stream) with a gas stream (here: hydrogen chloride gas stream) employed according to the invention are bubble columns or distillation columns configured as tray columns. Bubble columns may be used either without internals or, preferably, with internals (for example static systems such as perforated plates or dynamic mixer systems) to reduce backmixing and/or to allow and/or assist redispersion of the liquid phase and the gas phase. Static and dynamic systems may optionally also be combined.

The hydrogen chloride treatment according to the invention may be performed in countercurrent or in cocurrent in bubble columns for example In the terminology of the present invention, countercurrent is to be understood as meaning that in the case of a vertically arranged bubble column the liquid product stream passes through the apparatus from top to bottom while the gas stream passes through the apparatus from bottom to top. In the terminology of the present invention cocurrent is to be understood as meaning that in the case of a vertically arranged bubble column both the liquid product stream and the gas stream pass through the apparatus from below. The countercurrent mode is preferred when performing the hydrogen chloride treatment according to the invention in bubble columns

In any case it is preferable to perform the treatment with the gaseous hydrogen chloride stream in step (VII) at a temperature of the product stream to be treated or of the product mixture to be treated in the range from 70° C. to 135° C., preferably 80° C. to 135° C., particularly preferably 90° C. to 135° C., and at a temperature of the hydrogen chloride stream employed in the range from 20° C. to 135° C., preferably 25° C. to 135° C., particularly preferably 30° C. to 135° C. It is further preferable, in particular in connection with maintaining the abovementioned temperatures, to perform the hydrogen chloride treatment according to the invention isothermally, i.e. by keeping the temperature as constant as possible by thermostatting (i.e. according to the heat of reaction altogether resulting from the physical and chemical processes taking place, by addition of heat by heating or removal of heat by cooling, here in particular by addition of heat, optionally assisted by insulation of the apparatus employed against heat losses to the surroundings).

The treatment with the gaseous hydrogen chloride stream in step (VII) is carried out at ambient pressure or at a pressure which is elevated relative to ambient pressure, preferably at a pressure in the range of ambient pressure, in particular 1.01 bar_{(abs.)}, to 5.00 bar_{(abs.)}, particularly preferably at a pressure of 1.50 bar_{(abs.)} to 3.50 bar_{(abs.)}.

Irrespective of the precise nature of the process management the present invention provides for establishing a contact time of the gaseous hydrogen chloride stream with the product stream to be treated in the range from 1 min to less than 30 min, preferably 1 min to 25 min, particularly preferably 1 min to 20 min, very particularly preferably 2 min to 20 min, exceptionally preferably 4 min to 20 min.

The hydrogen chloride treatment according to the invention may in principle be performed with hydrogen chloride from a very wide variety of sources. In addition to pure hydrogen chloride, the use of hydrogen chloride co-produced in chemical reactions, in particular in the MDI preparation itself, is also conceivable. In the latter case in particular, the hydrogen chloride may still contain small amounts of phosgene, in particular in a mass fraction based on its total mass in the range from 1 ppm to 10 000 ppm, preferably from 10 ppm to 10 000 ppm, particularly preferably in the range from 10 ppm to 1000 ppm. It has now been found that, entirely surprisingly, this phosgene content has no negative consequences, neither on the actual hydrogen chloride treatment nor on the quality of the MDI prepared.

In the preferred embodiment of the invention where step (VI) is performed, the sixth gaseous product stream obtained in this step may advantageously be partially or completely used as the gaseous hydrogen chloride stream in step (VII) (wherein further hydrogen chloride may be but need not be added). This has the great advantage that the use of hydrogen chloride from external sources (sources external to the MDI process) is minimized or even becomes completely dispensable.

Irrespective of the precise source of the hydrogen chloride and the type of the product stream or reaction mixture to be treated, it is preferable to establish in step (VII) a molar ratio of hydrogen chloride present in the gaseous hydrogen chloride stream to be employed in step (VII) to isocyanate groups present in the reaction mixture or product stream to be treated in step (VII), n(HCl)/n(NCO), in the range from 0.1 to 2.0, preferably 0.1 to 1.0, particularly preferably 0.1 to 0.5.

The particularly preferred embodiment of the invention comprising performing the hydrogen chloride treatment according to the invention in variant (VII)(a) is shown in FIG. 2. The reference symbols, also used in FIG. 1, have the same definitions as therein. The further reference symbols are as follows:

Apparatuses:
7000: Apparatus (=tray column) for treating (=passing through) the first liquid product stream (30) with a hydrogen chloride gas stream in countercurrent;
7100: Trays;

Material Streams:
30′: First liquid product stream (30) treated with hydrogen chloride gas;
410: Hydrogen chloride gas stream;
420: Hydrogen chloride gas stream remaining after passing through column 7000. Said stream is preferably combined (not shown in FIG. 2) with the streams 20, 40 (if present) and 60, so that stream 420 becomes a constituent of stream 100.

Overall, the invention therefore exhibits the following advantages:
Significant reduction in color values.
Significant reduction in chlorine content (expressed as total chlorine content and/or as hydrolyzable chlorine).
Contrary to the prior art it is not necessary to separate the phosgene from the liquid phase prior to the treatment with hydrogen chloride.
It is not necessary to use pure hydrogen chloride - on the contrary process streams containing hydrogen chloride having a residual content of phosgene are also suitable. There is therefore no need for complex and costly cleaning of the hydrogen chloride gas stream or the use of externally supplied, pure hydrogen chloride.
In the case of the preferred variant (VII) (a), the low thermal pre-load on the material additionally minimizes the tendency for secondary reactions which could otherwise lead to undesired (sometimes also color-imparting) byproducts. A marked color improvement and reduction of the chlorine content in the end product are observed.
In the preferred variant (VII) (a) at least a portion of the phosgene is stripped out of the liquid phase by hydrogen chloride, thus at least partially relieving subsequent process steps of the task of phosgene separation.

### EXAMPLES

The examples which follow employed two different apparatuses for hydrogen chloride treatment and these are described below.

A) Tray Column (Reactive Rectification):

The column made of glass having an internal diameter of 50 mm and a length of 700 mm had been provided with 10 valve trays. The total liquid holdup on the trays of the column was about 200 ml. The tray column was operated at atmospheric pressure and was wrapped with an electrical heating tape to avoid heat radiation to the environment. The temperature of this trace heating was set to 120° C. The liquid input stream was introduced to the column above the uppermost tray while the gaseous input stream was supplied to the column below the lowermost tray (the tray column was thus operated in countercurrent in the terminology of the present invention).

Both input streams were temperature-controlled using heat exchangers before they entered the column The temperature of the gas stream was about 60° C. to 80° C. The temperature of the liquid input stream was about 125° C.

The gaseous output stream was withdrawn at the top of the column and passed through a heat exchanger to collect condensable constituents (essentially monochlorobenzene, MCB). The liquid output stream was withdrawn at the bottom of the column below the addition point of the gaseous input stream and collected in a double-walled vessel temperature-controlled to 60° C., from which the samples for further workup and analysis were taken.

B) Bubble Column

The double-walled bubble column made of glass having an internal diameter of 50 mm and a length of 700 mm had been provided with a total of 10 perforated plates, each having 2.5% free area (hole area) and a ring sparger. The lower portion of the bubble column up to a height of 150 mm was separated off by a perforated plate and served as a temperature control zone. The ring sparger was arranged 50 mm thereabove and the further 9 perforated plates were arranged above that, each at a distance of 50 mm from the other. The volume of the bubble column above the ring sparger was about 1000 ml. The double wall used for temperature-controlling the bubble column (heat transfer medium: silicone oil) was divided into two sections, whose temperature was adjustable independently of one another. The lower section comprised the area below the perforated plate arranged below the ring sparger and the upper section comprised the larger portion of the bubble column thereabove.

The gaseous input stream was supplied to the bubble column via the ring sparger at a temperature of about 20° C. to 25° C. (ambient temperature). The gaseous output stream was withdrawn at the top of the bubble column and passed through a heat exchanger to collect condensable constituents (essentially MCB).

The bubble column was operated in cocurrent. The liquid input stream was initially temperature-controlled to 100° C. using a heat exchanger and then introduced into the bubble column at the bottom of the temperature control zone below the feed point of the gaseous input stream at the bottom of the column The liquid output stream was withdrawn at the top of the bubble column about 70 mm above the uppermost perforated plate and collected in a double-walled vessel temperature-controlled to 60° C., from which the samples for further workup and analysis were taken. (Operation of the bubble column in countercurrent is also quite possible. To this end, the liquid input stream (after temperature-controlling to 100° C. using a heat exchanger as in cocurrent operation) may be introduced to the bubble column about 70 mm above the uppermost perforated plate. The liquid output stream may be withdrawn at the bottom of the column below the feed point for the gaseous input stream at the bottom of the temperature control zone and subjected to further treatment as described for cocurrent operation.)

The bubble column was operated at elevated pressure which was monitored using a manometer. Pressure maintenance was effected manually via needle valves in the outlet for the liquid output stream and the gaseous output stream. (However, operation of the bubble column at ambient pressure is also quite possible.)

Irrespective of the apparatus used, the gaseous input materials were either hydrogen chloride (from a compressed gas bottle), nitrogen or phosgene, or mixtures of these gases. The liquid input materials used were samples obtained in the course of the phosgenation of MDA in MCB, i.e.—in the terminology of the present invention—a first liquid product stream (i.e. the output from a phosgenation reactor, inter alia containing MCB, MDI, corresponding carbamoyl chlorides and phosgene) or a third liquid product stream (i.e. the output from a dephosgenation stage, inter alia containing MCB and MDI—the stream was practically free from carbamoyl chloride and phosgene). (More details may be apparent from the specific descriptions of the individual examples in table 1.)

The samples obtained after the treatment—and untreated reference samples—were worked up according to a standardized procedure. Most of the MCB solvent present in the sample was initially distilled off under water jet vacuum (approx. 20 mbar_{(abs)}) wherein the bottoms temperature of 100° C. was not exceeded. The liquid bottoms (essentially MDI) were then transferred into a smaller distillation apparatus in which the solvent residues were distilled off under reduced pressure (about 2 to 3 mbar_{(abs)}), specifically up to a bottoms temperature of 190° C. The distillation was then discontinued and the distillation bottoms (MDI) cooled to room temperature over just a few minutes.

The product obtained (MDI) was subjected to analytical characterization by means of the following parameters: NCO value, viscosity, color values (extinction at 430 nm and 520 nm) and also total chlorine content in a portion of the tests.

The NCO value was determined by reacting a sample of the isocyanate with excess di-n-butylamine, followed by back titration of the excess amine with a hydrochloric acid standard solution.

Dynamic viscosity was measured at 25° C. using a falling ball viscometer.

To determine the color values (E430, E520), a solution of 1.00 g of isocyanate in 50 ml of MCB was prepared. A portion of this solution was transferred to a cuvette with a 10 mm path length and the extinction at 430 nm or 520 nm was measured against MCB as a reference using a Hach Lange LICO 690 spectral colorimeter.

The total chlorine content (total chlorine, TC) was determined by X-ray fluorescence analysis.

TABLE 1
Comparison of the test conditions and results of the examples

| | | |
|---|---|---|
| [symbol] | [symbol] | [symbol] |

AP = ambient pressure (about 1 bar_{(abs.)});
n.d. = not determined [symbol]
indicates data missing or illegible when filed

The results of Examples 1 to 16 demonstrate the following:

The treatment according to the invention with gaseous hydrogen chloride results in a marked improvement in the color of the MDI obtained (lowering of the extinction values at 430 nm and 520 nm). A reduction of the total chlorine content is also achieved. It is essential that hydrogen chloride gas is used, since comparative tests have shown that the use of an inert gas such as nitrogen fails to show any positive effect. In contrast to what is stated in parts of the prior art, the process according to the invention also allows the presence of a certain proportion of phosgene in the gaseous hydrogen chloride employed and/or in the product stream or reaction mixture to be treated: The examples show that it is by no means necessary to separate off excess phosgene from the phosgenation before the treatment with gaseous hydrogen chloride is performed. On the contrary, the result is even improved when the hydrogen chloride treatment is performed on the liquid crude product of the phosgenation.

1. A process for preparing di- and polyisocyanates of the diphenylmethane series, comprising:
(I) reacting di- and polyamines of the diphenylmethane series with phosgene in the presence of a solvent in a phosgenation reactor to obtain (a) a first gaseous product stream containing hydrogen chloride and phosgene and (b) a first liquid product stream containing di- and polyisocyanates of the diphenylmethane series, phosgene and solvent;
(II) optionally reacting the first liquid product stream in a reactor for carbamoyl chloride cleavage to form in the reactor for carbamoyl chloride cleavage a first reaction mixture which is separated into (a) a second gaseous product stream containing hydrogen chloride and phosgene and (b) a second liquid product stream containing isocyanate, phosgene and solvent;
(III) separating phosgene from the first liquid product stream or, if step (II) is performed, from the second liquid product stream in a dephosgenation apparatus to form in the dephosgenation apparatus a second reaction mixture which is separated into (a) a third gaseous product stream containing hydrogen chloride and phosgene and (b) a third liquid product stream containing isocyanate and solvent;
(IV) separating solvent from the third liquid product stream to obtain (a) a fourth gaseous product stream containing solvent and (b) a fourth liquid product stream containing diisocyanates of the diphenylmethane series, wherein the separation of the solvent is not followed by an inert gas treatment of the fourth liquid product stream;
(V) optionally separating a portion of the diisocyanates of the diphenylmethane series from the fourth liquid product stream to obtain (a) a fifth gaseous product stream containing diisocyanates of the diphenylmethane series, which is condensed, and (b) a fifth liquid product stream enriched in polyisocyanates of the diphenylmethane series relative to the fourth liquid product stream;
(VI) optionally separating phosgene from the first and third gaseous product stream, if performing step (II) from the first, second and third gaseous product stream, to obtain (a) a sixth gaseous product stream containing hydrogen chloride and (b) a sixth liquid product stream containing phosgene;
wherein
(VII) (a) the first liquid product stream obtained in step (I) is treated with a gaseous hydrogen chloride stream and then supplied directly to step (II) or step (III),
(b) the first reaction mixture formed in the reactor for carbamoyl chloride cleavage from step (II) is treated with a gaseous hydrogen chloride stream,
(c) the second liquid product stream obtained in step (II) is treated with a gaseous hydrogen chloride stream and then supplied directly to step (III),
(d) the second reaction mixture formed in the dephosgenation apparatus from step (III) is treated with a gaseous hydrogen chloride stream
or
(e) the third liquid product stream obtained in step (III) is treated with a gaseous hydrogen chloride stream and then supplied directly to step (IV),
wherein the treatment with the gaseous hydrogen chloride stream is performed in a single stage in a bubble column or in a tray column, wherein a contact time of the gaseous hydrogen chloride stream with the liquid product stream to be treated or the reaction mixture to be treated in the range from 1 min to less than 30 min is established.

2. The process as claimed in claim 1, wherein step (VII) is performed according to (VII)(a), (VII)(c) or (VII)(e).

3. The process as claimed in claim 1, wherein step (VII) is performed according to (VII)(a) or (VII)(c).

4. The process as claimed in claim 1, wherein the third liquid product stream contains phosgene in a mass fraction based on its total mass in the range from 0.001 ppm to 1000 ppm.

5. The process as claimed in claim 1, wherein the gaseous hydrogen chloride stream employed in step (VII) contains phosgene in a mass fraction based on its total mass in the range from 1 ppm to 10 000 ppm.

6. The process as claimed in claim 1, wherein the process comprises step (VI) and wherein the sixth gaseous product stream is partially or completely used as the gaseous hydrogen chloride stream in step (VII).

7. The process as claimed in claim 1, wherein the molar ratio of hydrogen chloride present in the gaseous hydrogen chloride stream employed in step (VII) to isocyanate groups present in the reaction mixture or product stream treated in step (VII), n(HCl)/n(NCO), is set to a value in the range from 0.1 to 2.0.

8. The process as claimed in claim 1, wherein the gaseous hydrogen chloride stream and the product stream to be treated or the reaction mixture to be treated are run in countercurrent in step (VII).

9. The process as claimed in claim 1, wherein the gaseous hydrogen chloride stream and the product stream to be treated or the reaction mixture to be treated are run in cocurrent in step (VII).

10. The process as claimed in claim 1, wherein the treatment with the gaseous hydrogen chloride stream in step (VII) is performed at a temperature of the product stream to be treated or of the product mixture to be treated in the range from 70° C. to 135° C. and at a temperature of the gaseous hydrogen chloride stream employed in the range from 20° C. to 135° C.

11. The process as claimed in claim 1, wherein the treatment with the gaseous hydrogen chloride stream in step (VII) is performed isothermally.

12. The process as claimed in claim 1, wherein a contact time of the gaseous hydrogen chloride stream with the product stream to be treated or the reaction mixture to be treated in the range from 1 min to 25 min is established in step (VII).

13. The process as claimed in claim 12, wherein a contact time of the gaseous hydrogen chloride stream with the product stream to be treated or the reaction mixture to be treated in the range from 1 min to 20 min is established in step (VII).

14. The process as claimed in claim 1, wherein the treatment with the gaseous hydrogen chloride stream in step (VII) is carried out at a pressure in the range from ambient pressure to 5.0 bar_{(abs.)}.

15. The process as claimed in claim 14, wherein the treatment with the gaseous hydrogen chloride stream in step (VII) is carried out at a pressure in the range from ambient pressure to 3.5 bar_{(abs.)}.
